(19) United States
(12) Patent Application Publication
(10) Pub. No.: US 2020/0397329 A1
(43) Pub. Date: Dec. 24, 2020

(54) METHODS AND SYSTEMS FOR TRANSMURAL TISSUE MAPPING

(71) Applicant: St. Jude Medical, Cardiology Division, Inc., St. Paul, MN (US)

(72) Inventors: Jan MANGUAL, Rho (IT); Craig MARKOVITZ, Leipzig (DE); Chunlan JIANG, Crystal, MN (US); Wenwen LI, San Jose, CA (US); Louis-Philippe RICHER, Montreal (CA); Cyrille CASSET, Saint Selve (FR); Luke MCSPADDEN, Los Angeles, CA (US)

(21) Appl. No.: 16/972,000

(22) PCT Filed: Mar. 5, 2019
(86) PCT No.: PCT/US2019/020683

§ 371 Date: Aug. 21, 2020

Related U.S. Application Data
(60) Provisional application No. 62/639,146, filed on Mar. 6, 2018.

(51) Int. Cl. A61B 5/042; A61B 5/00; A61B 5/107

(52) U.S. Cl. CPC A61B 5/0422; A61B 5/6852; A61B 5/4836; A61B 5/1072; A61B 5/746

(57) ABSTRACT

Systems and methods for measuring transmural activation times between an endocardial surface and an epicardial surface are provided. A system includes at least one catheter including at least one electrode, the at least one catheter configured to acquire electrogram data and positioning data proximate at least one of the endocardial surface and the epicardial surface. The system further includes a computing device communicatively coupled to the at least one catheter, the computing device configured to determine transmural activation times based on the acquired electrogram data and positioning data.

### CROSS REFERENCE TO RELATED APPLICATIONS

This application claims priority to provisional application Ser. No. 62/639,146, filed Mar. 6, 2018, which is incorporated herein by reference in its entirety.

### FIELD OF THE DISCLOSURE

This disclosure relates to systems and methods for generating cardiac models, and more particularly, this disclosure relates to systems and methods for transmural tissue mapping.

### BACKGROUND

It is known that various computer-based systems and computer-implemented methodologies can be used to generate multi-dimensional surface models of geometric structures, such as, for example, anatomic structures. More specifically, a variety of systems and methods have been used to generate multi-dimensional surface models of the heart and/or particular portions thereof.

The human heart muscle routinely experiences electrical currents traversing its many surfaces and ventricles, including the endocardial surfaces. Just prior to each heart contraction, the heart muscle is said to “depolarize” and “repolarize,” as electrical currents spread across the heart and throughout the body. In healthy hearts, the surfaces and ventricles of the heart will experience an orderly progression of a depolarization wave. In unhealthy hearts, such as those experiencing atrial arrhythmia, including for example, ectopic atrial tachycardia, atrial fibrillation, and atrial flutter, the progression of the depolarization wave may not be so orderly. Arrhythmias may persist as a result of scar tissue or other obstacles to rapid and uniform depolarization. These obstacles may cause depolarization waves to repeat a circuit around some part of the heart. Atrial arrhythmia can create a variety of dangerous conditions, including irregular heart rates, loss of synchronous atrioventricular contractions, and stasis of blood flow, all of which can lead to a variety of ailments and even death.

Medical devices, such as, for example, electrophysiology (EP) catheters, are used in a variety of diagnostic and/or therapeutic medical procedures to correct such heart arrhythmias. Typically in a procedure, a catheter is manipulated through a patient's vasculature to a patient's heart, for example, and carries one or more electrodes that may be used for mapping, ablation, diagnosis, and/or to perform other functions. Once at an intended site, treatment may include delivery of radio frequency (RF) ablation, cryoablation, lasers, chemicals, high-intensity focused ultrasound, etc. to the heart tissue. An ablation catheter imparts such ablative energy to cardiac tissue to create a lesion in the cardiac tissue. This lesion disrupts undesirable electrical pathways and thereby limits or prevents stray electrical signals that lead to arrhythmias. As readily apparent, such treatment requires precise control of the catheter during manipulation to, from, and at the treatment site, which can invariably be a function of a user's skill level.

For complex arrhythmia ablation procedures, three-dimensional analysis of cardiac tissue is utilized. As technology has advanced, tools for adequate mapping and substrate identification have also evolved, providing physicians with a better understanding of the origin of arrhythmias, as well as their progression and diseased state. For example, intramural scar tissue may facilitate intramural or transmural reentry circuits, which may be detected by prolonged transmural activation intervals.

At least some known systems facilitate surface mapping of epicardial and endocardial tissue surfaces. However, information on transmural electrical propagation (i.e., propagation between the epicardial and endocardial surfaces) is generally not evaluated. Accordingly, to enable further evaluation of cardiac tissue, it would be desirable to record and analyze transmural activation times between corresponding epicardial and endocardial points.

### BRIEF SUMMARY OF THE DISCLOSURE

In one embodiment, the present disclosure is directed to a system for measuring transmural activation times between an endocardial surface and an epicardial surface. The system includes at least one catheter including at least one electrode, the at least one catheter configured to acquire electrogram data and positioning data proximate at least one of the endocardial surface and the epicardial surface. The system further includes a computing device communicatively coupled to the at least one catheter, the computing device configured to determine transmural activation times based on the acquired electrogram data and positioning data.

In another embodiment, the present disclosure is directed to a method for measuring transmural activation times between an endocardial surface and an epicardial surface. The method includes acquiring electrogram data and positioning data using at least one catheter, the at least one catheter including at least one electrode proximate at least one of the endocardial surface and the epicardial surface, and determining, using a computing device communicatively coupled to the at least one catheter, at least one transmural activation time based on the acquired electrogram data and positioning data.

In yet another embodiment, the present disclosure is directed to a computing device for measuring transmural activation times between an endocardial surface and an epicardial surface. The computing device is configured to receive electrogram data and positioning data from at least one catheter communicatively coupled to the computing device, the at least one catheter including at least one electrode proximate at least one of the endocardial surface and the epicardial surface, and determine at least one transmural activation time based on the acquired electrogram data and positioning data.

The foregoing and other aspects, features, details, utilities and advantages of the present disclosure will be apparent from reading the following description and claims, and from reviewing the accompanying drawings.

### BRIEF DESCRIPTION OF THE DRAWINGS

FIG. 1 is a schematic and diagrammatic view of a system for performing at least one of a diagnostic and a therapeutic medical procedure in accordance with present teachings.

FIG. 2 is an isometric view of a distal end of one embodiment of a medical device arranged in a spiral configuration.

FIG. 3 is an isometric view of a distal end of another embodiment of a medical device arranged in a basket configuration.

FIGS. 4A and 4B are isometric and side views, respectively, of a distal end of one embodiment of a medical device arranged in a matrix-like configuration.

FIG. 5 is a top view of a distal end of one embodiment of a medical device wherein the medical device is a radio frequency (RF) ablation catheter.

FIG. 6 is a schematic and diagrammatic view of one embodiment of a visualization, navigation, and mapping subsystem that may be used with the system shown in FIG. 1.

FIG. 7 is a schematic view of one embodiment of system for measuring transmural activation between an endocardial surface and an epicardial surface.

FIG. 8 is an enlarged view of a portion of the cardiac wall shown in FIG. 7.

FIG. 9A is an electrogram measured using the system shown in FIG. 7 showing an endocardial activation time.

FIG. 9B is an electrogram measured using the system shown in FIG. 7 showing an epicardial activation time.

FIG. 10 is schematic view of an alternative embodiment of a system for measuring transmural activation between an endocardial surface and an epicardial surface.

FIG. 11 is an electrogram measured using the system shown in FIG. 10.

FIG. 12 is an electrogram measured using the system shown in FIG. 10.

Corresponding reference characters indicate corresponding parts throughout the several views of the drawings.

### DETAILED DESCRIPTION OF THE DISCLOSURE

The disclosure provides systems and methods for measuring transmural activation times between an endocardial surface and an epicardial surface. At least one catheter including at least one electrode is acquires electrogram data and positioning data proximate at least one of the endocardial surface and the epicardial surface. A computing device communicatively coupled to the at least one catheter determines transmural activation times based on the acquired electrogram data and positioning data, as described herein.

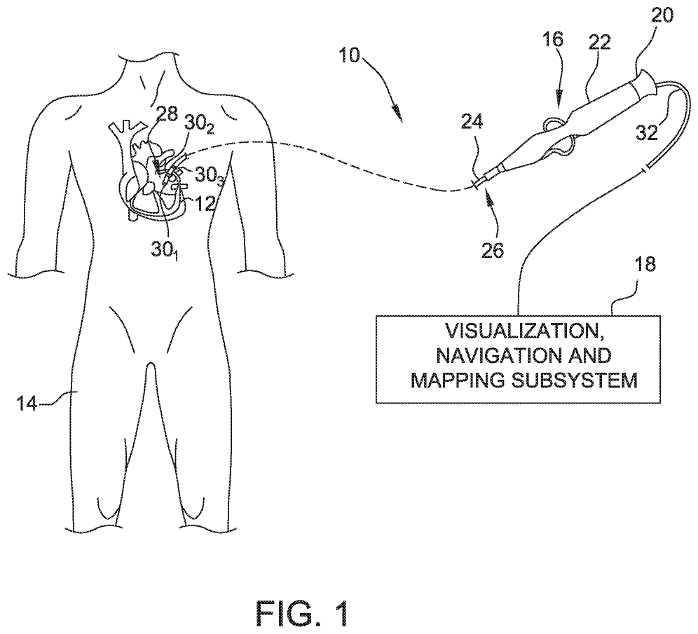

Referring now to the drawings wherein like reference numerals are used to identify identical components in the various views, FIG. 1 illustrates one exemplary embodiment of a system 10 for performing one or more diagnostic and/or therapeutic functions on or for a tissue 12 of a body 14. In an exemplary embodiment, tissue 12 includes heart or cardiac tissue within a human body 14. It should be understood, however, that system 10 may find application in connection with a variety of other tissues within human and non-human bodies, and therefore, the present disclosure is not meant to be limited to the use of system 10 in connection with only cardiac tissue and/or human bodies.

System 10 may include a medical device (e.g., a catheter 16) and a subsystem 18 for the visualization, navigation, and/or mapping of internal body structures (hereinafter referred to as the “visualization, navigation, and mapping subsystem 18”, “subsystem 18”, or “mapping system”).

In this embodiment, medical device includes a catheter 16, such as, for example, an electrophysiology catheter. In other exemplary embodiments, medical device may take a form other than catheter 16, such as, for example and without limitation, a sheath or catheter-introducer, or a catheter other than an electrophysiology catheter. For clarity and illustrative purposes only, the description below will be limited to embodiments of system 10 wherein medical device is a catheter (catheter 16).

Catheter 16 is provided for examination, diagnosis, and/or treatment of internal body tissues such as tissue 12. Catheter 16 may include a cable connector 20 or interface, a handle 22, a shaft 24 having a proximal end 26 and a distal end 28 (as used herein, “proximal” refers to a direction toward the end of catheter 16 near handle 22, and “distal” refers to a direction away from handle 22), and one or more sensors, such as, for example and without limitation, a plurality of electrodes 30 (i.e., 30₁, 30₂, . . . , 30_{N}), mounted in or on shaft 24 of catheter 16 at or near distal end 28 of shaft 24. The sensors may include, for example, impedance electrodes.

In this embodiment, each electrode 30 is configured to both acquire electrophysiological (EP) data corresponding to tissue 12, and to produce signals indicative of its three-dimensional (3-D) position (hereinafter referred to as “positioning data”). In another embodiment, catheter 16 may include a combination of electrodes 30 and one or more positioning sensors (e.g., electrodes other than electrodes 30 or magnetic sensors (e.g., coils)). In one such embodiment, electrodes 30 are configured to acquire EP data relating to tissue 12, while the positioning sensor(s) is configured to generate positioning data indicative of the 3-D position thereof, which may be used to determine the 3-D position of each electrode 30. In other embodiments, catheter 16 may further include other conventional components such as, for example and without limitation, steering wires and actuators, irrigation lumens and ports, pressure sensors, contact sensors, temperature sensors, additional electrodes and corresponding conductors or leads, and/or ablation elements (e.g., ablation electrodes, high intensity focused ultrasound ablation elements, and the like).

Connector 20 provides mechanical and electrical connection(s) for one or more cables 32 extending, for example, from visualization, navigation, and mapping subsystem 18 to one or more electrodes 30 or the positioning sensor(s) mounted on catheter 16. In other embodiments, connector 20 may also provide mechanical, electrical, and/or fluid connections for cables extending from other components in system 10, such as, for example, an ablation system and a fluid source (when catheter 16 includes an irrigated catheter). Connector 20 is disposed at a proximal end of catheter 16.

Handle 22 provides a location for a user to hold catheter 16 and may further provide means for steering or guiding shaft 24 within body 14. For example, handle 22 may include means to manipulate one or more steering wires extending through catheter 16 to distal end 28 of shaft 24 to steer shaft 24. It will be appreciated by those of skill in the art that the construction of handle 22 may vary. In other embodiments, the control of catheter 16 may be automated such as by being robotically driven or controlled, or driven and controlled by a magnetic-based guidance system. Accordingly, catheters controlled either manually or automatically are both within the spirit and scope of the present disclosure.

Shaft 24 is an elongate, tubular, and flexible member configured for movement within body 14. Shaft 24 supports, for example and without limitation, electrodes 30, other electrodes or positioning sensors mounted thereon, associated conductors, and possibly additional electronics used for signal processing or conditioning. Shaft 24 may also permit transport, delivery and/or removal of fluids (including irrigation fluids, cryogenic ablation fluids, and body fluids), medicines, and/or surgical tools or instruments. Shaft 24, which may be made from conventional materials such as polyurethane, defines one or more lumens configured to house and/or transport electrical conductors, fluids, or surgical tools. Shaft 24 may be introduced into a blood vessel or other structure within body 14 through a conventional introducer. Shaft 24 may then be steered or guided through body 14 to a desired location such as tissue 12.

Distal end 28 of shaft 24 may be the main portion of catheter 16 that contains electrodes 30 or other sensors for acquiring EP data and positioning data. As described above, in one embodiment, electrodes 30 may be configured to acquire both EP data and positioning data. In another embodiment, and as will be described in greater detail below, electrodes 30 may be configured to acquire EP data while one or more positioning sensors may be configured to acquire positioning data, which may then be used to determine the respective positions of electrodes 30. Regardless of whether the positioning data is acquired by electrodes 30 or by positioning sensors, distal end 28 may be arranged in a number of configurations that facilitate the efficient acquisition, measurement, collection, or the like of EP data from tissue 12.

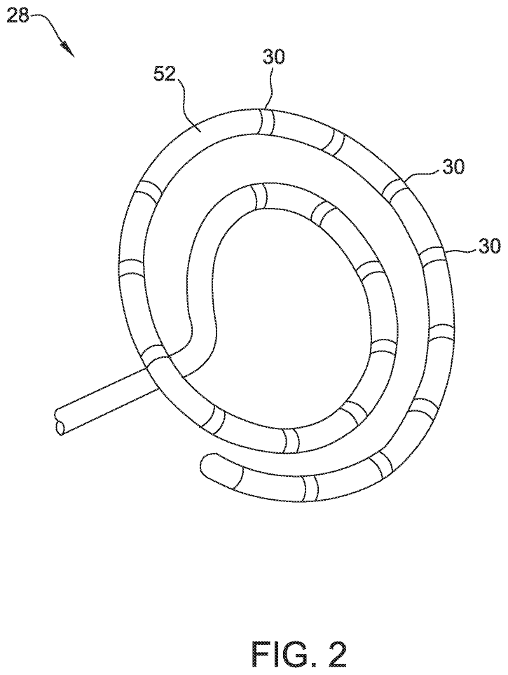

In one embodiment, as shown in FIG. 2, distal end 28 may be arranged in a spiral configuration. In this embodiment, the spiral configuration may be generally planar and may contain a high density of electrodes 30 for taking unipolar or bipolar measurements of EP data from tissue 12. Unipolar measurements may generally represent the electrical voltage perceived at each electrode. Bipolar measurements, though, may generally represent the electrical potential between any pair of electrodes. And as one skilled in the art will recognize, bipolar measurements may be computed from unipolar measurements. Moreover, electrodes 30 may be disposed in or along distal end 28 in a known spatial configuration such that the distances between electrodes 30 are known. The diameters of the loops, such as loop 52, may vary from one embodiment to another. In one embodiment, the diameter of the outermost loop is twenty millimeters. In an alternative embodiment, the spiral configuration may contain multiple spiral loops.

There are many advantages to placing a high density of electrodes 30 on the spiral configuration or at distal end 28 of catheter 16. Because the distribution of electrodes 30 is dense, and because of the multitude of possible unipolar and bipolar comparisons of electrodes 30, the spiral configuration may be ideal for creating high definition (HD) surface maps representative of electrical activity on tissue 12.

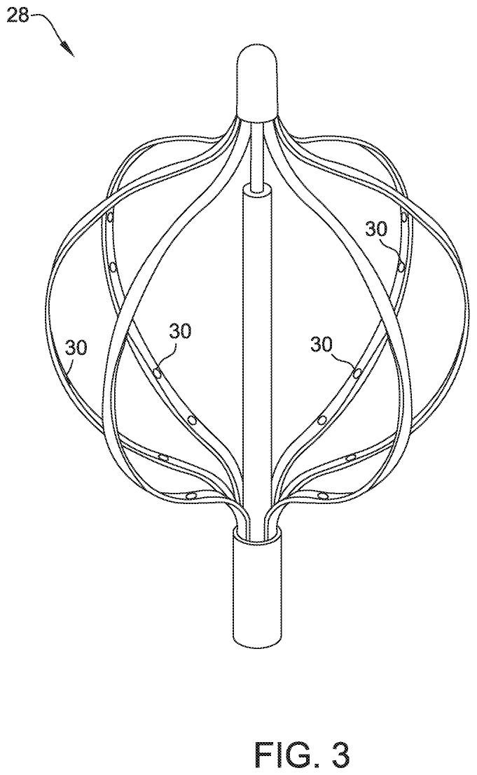

In another embodiment, as shown in FIG. 3, distal end 28 may be arranged in a basket configuration. The basket configuration, or a similar configuration with a generally cylindrical array of electrodes 30, may contain a high density of electrodes 30. In one embodiment, electrodes 30 may be non-contact electrodes that generally need not be in contact with tissue 12 to measure EP data. In another embodiment, electrodes 30 may include both contact and non-contact electrodes.

Such non-contact electrodes may be used for unipolar analyses. It may be advantageous to analyze unipolar EP data since a unipolar electrogram morphology may provide more information regarding colliding wavefronts (presence of “R” waves in the QRS Complex known in the art), short radius reentry wavefronts (presence of the sinusoid waveform), and source wavefronts (a “QS” morphology on the electrogram at the onset of depolarization). In general, a depolarization wavefront is a group of electrical vectors that traverse tissue 12 of body 14. Depolarization wavefronts may vary in pattern, size, amplitude, speed, and the like. And some depolarization wavefronts may be relatively orderly while others may be relatively, or even entirely, disorderly.

In another embodiment, however, bipolar EP data may provide better spatial localization data, better depolarization wave directionality indications, and better alternating current (AC) electrical noise rejection. With bipolar EP data, a pair of electrodes 30 (commonly referred to as “poles” or “bi-poles”) may be spaced apart, but positioned relatively close together with respect to electric fields caused by other remote parts of body 14. Thus, effects from remote electric fields may be negated since electrodes 30 are positioned close to one another and experience similar effects from the distant electric field.

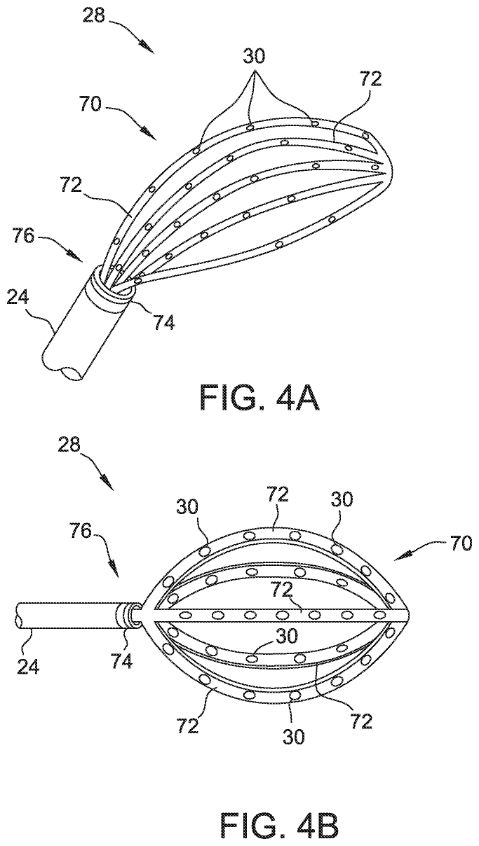

In yet another embodiment of the distal end 28 shown in FIGS. 4A and 4B, a matrix-like configuration may also be provided with a high density of electrodes 30. FIG. 4A shows an isometric view of the matrix-like configuration, while FIG. 4B shows a side view. The matrix-like configuration may have a number of splines 72 arranged side by side, with each spline 72 having at least one electrode 30 mounted thereon. Longer splines may contain more electrodes 30 to maintain a consistent electrode density throughout the matrix-like configuration.

In the embodiment shown in FIGS. 4A and 4B, the matrix-like configuration may be cupped, almost as if to have a slight scoop as seen in FIG. 4A. In another embodiment (not shown), the matrix-like configuration may be substantially flat or planar, without any scoop-like feature. While both embodiments may facilitate data measurements from tissue 12, the matrix-like configuration shown in FIG. 4A in particular may be used to acquire at least some non-contact measurements. Another possible use of the matrix-like configuration would be to help diagnose arrhythmias and direct epicardial ablation therapies in the pericardial space.

In one embodiment, the matrix-like configuration along with other configurations of distal end 28 may collapse to a streamlined profile for insertion, manipulation, and removal from body 14. In addition, or in the alternative, distal end 28 may be at least partially concealed and transported within shaft 24 when not collecting data or performing a procedure. Shaft 24 may be more streamlined than distal end 28, and therefore may provide a better vehicle for transporting distal end 28 to and from tissue 12. Once at the intended site, distal end 28 may be deployed from shaft 24 to perform the intended procedures. Likewise, after the procedures are performed, distal end 28 may be re-concealed, at least in part, within shaft 24 for removal from body 14.

One exemplary way in which the matrix-like configuration is collapsible into a streamlined profile or fully or partially deployable is to allow outer splines 72 to translate modestly within shaft 24 while anchoring innermost splines 72 to shaft 24 at a point 74 at distal end 28 thereof. Moreover, for enhanced functionality, a joint 76 may be incorporated near point 74, either for providing flexibility or for selectively deflecting distal end 28, thereby allowing distal end 28 better access to tissue 12.

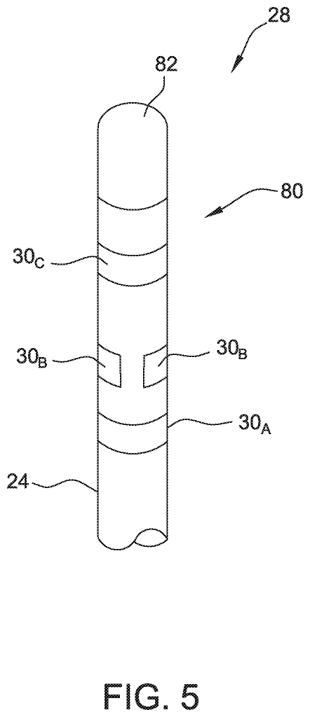

Another exemplary embodiment of a high-density electrode catheter is illustrated in FIG. 5. In this embodiment, distal end 28 includes an ablation tip 80, and may be well suited for enhancing radio frequency (RF) ablation procedures. More particularly, the arrangement may allow for the provision of rapid positioning feedback and may also enable updates to be made to HD surface maps as the ablative procedures are being performed.

With continued reference to FIG. 5, in an exemplary embodiment wherein visualization, navigation, and mapping subsystem 18 is an electric field-based system, distal end 28 may include a proximal ring electrode 30_{A} positioned close to, yet spaced apart from, a series of spot or button electrodes 30_{B}. Proximal ring electrode 30_{A} and spot electrodes 30_{B} may be used to acquire both EP data and positioning data. Spaced further distally from the spot electrodes 30_{B}, a distal ring electrode 30_{C} may be disposed in or on shaft 24 so that bipolar measurements of EP data may be made between the spot electrodes 30_{B} and the distal ring electrode 30_{C}. Finally, distal end 28 further includes an ablation electrode 82 for performing ablation therapies, such as, for example and without limitation, RF ablation therapies.

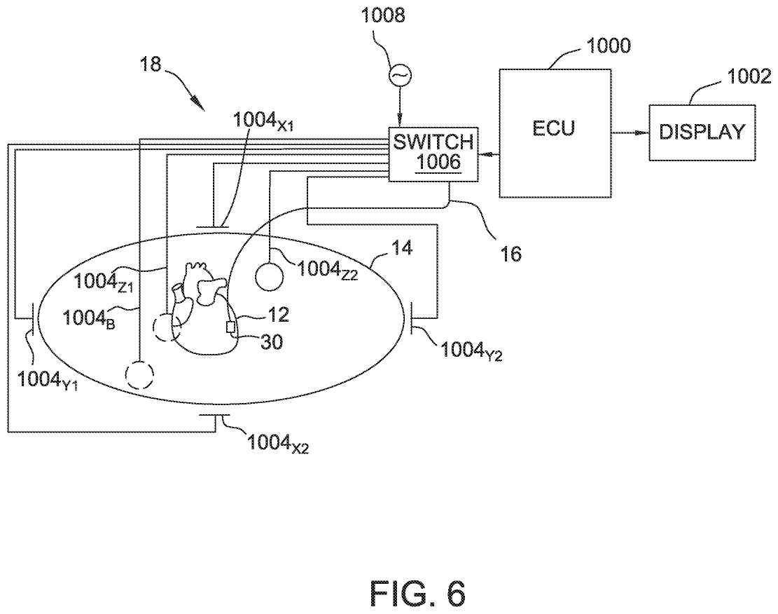

Visualization, navigation, and mapping subsystem 18 may determine the positions of proximal ring electrode 30_{A} (or a geometric center thereof), the spot electrodes 30_{B}, and distal ring electrode 30_{C} (or a geometric center thereof) in the same manner as the position(s) of the electrode(s) 30 shown in FIG. 6, as will be described in greater detail below. Based on these positions and/or the known configuration of distal end 28 (e.g., the spacing of the various electrodes), the position of ablation electrode 82 may also be determined and, in certain embodiments, projected onto a geometrical anatomical model.

By incorporating at least three non-co-linear electrodes as is illustrated, for example, in FIG. 5, rotational information about distal end 28 (referred to as “orientation”) may be calculated. Hence six degrees of freedom (three for position and three for orientation) may be determined for ablation tip 80 of catheter 16. Knowing the position and orientation of distal end 28 allows for a much simpler registration of coordinates into a body coordinate system, as opposed to a coordinate system with respect to the catheter itself.

In some embodiments, visualization, navigation, and mapping subsystem 18 includes a magnetic field-based system. For example visualization, navigation, and mapping subsystem 18 may include an electrical field- and magnetic field-based system such as the EnSite™ Precision™ system commercially available from Abbott Laboratories, and generally shown with reference to U.S. Pat. No. 7,263,397 entitled “Method and Apparatus for Catheter Navigation and Location and Mapping in the Heart”, the entire disclosure of which is incorporated herein by reference. In such embodiments, distal end 28 may include at least one magnetic field sensor—e.g., magnetic coils (not shown). If two or more magnetic field sensors are disposed near ablation electrode 82, a full six-degree-of-freedom registration of magnetic and spatial coordinates could be accomplished without having to determine orthogonal coordinates by solving for a registration transformation from a variety of positions and orientations. Further benefits of such a configuration may include advanced dislodgement detection and deriving dynamic field scaling since they may be self-contained.

In yet another embodiment of distal end 28 illustrated in FIG. 5, distal ring electrode 30_{C} may be omitted and spot electrodes 30_{B} may be located in its place. As a result, spot electrodes 30_{B} would be closer to ablation electrode 82, which would provide positioning coordinates closer to ablation electrode 82. This in turn may provide for more accurate and precise calculation of the position of ablation electrode 82. Additionally, just as if the distal ring electrode 30_{C} were still in place, a mean signal from the spot electrodes 30_{B} and the proximal ring electrode 30_{A} could still be used to obtain bipolar EP data.

With reference to FIGS. 1 and 6, the visualization, navigation, and mapping subsystem 18 will now be described. The visualization, navigation, and mapping subsystem 18 is provided for visualization, navigation, and/or mapping of internal body structures and/or medical devices. In an exemplary embodiment, the subsystem 18 may contribute to the functionality of the system 10 in two principal ways. First, the subsystem 18 may provide the system 10 with a geometrical anatomical model representing at least a portion of the tissue 12. Second, the subsystem 18 may provide a means by which the position coordinates (x, y, z) of the electrodes 30 (or generally, sensors) may be determined as they measure EP data for analyses performed as part of the system 10. In certain embodiments, positioning sensors (e.g., electrical-field based or magnetic-field based) that are fixed relative to the electrodes 30 are used to determine the position coordinates. The positioning sensors provide the subsystem 18 with positioning data sufficient to determine the position coordinates of the electrodes 30. In other embodiments, position coordinates may be determined from the electrodes 30 themselves by using, for example, voltages measured by the electrodes 30.

Visualization, navigation, and mapping subsystem 18 may utilize an electric field-based system, such as, for example, the ENSITE NAVX™ system commercially available from Abbott Laboratories, and as generally shown with reference to U.S. Pat. No. 7,263,397 titled “Method and Apparatus for Catheter Navigation and Location and Mapping in the Heart,” the entire disclosure of which is incorporated herein by reference, or the ENSITE VELOCITY™ system running a version of the NAVX™ software.

In other exemplary embodiments, subsystem 18 may utilize systems other than electric field-based systems. For example, subsystem 18 may comprise a magnetic field-based system such as the CARTO™ system commercially available from Biosense Webster, and as generally shown with reference to one or more of U.S. Pat. No. 6,498,944 entitled “Intrabody Measurement”; U.S. Pat. No. 6,788,967 entitled “Medical Diagnosis, Treatment and Imaging Systems”; and U.S. Pat. No. 6,690,963 entitled “System and Method for Determining the Location and Orientation of an Invasive Medical Instrument,” the disclosures of which are incorporated herein by reference in their entireties.

In yet another exemplary embodiment, subsystem 18 may include a magnetic field-based system such as the GMPS system commercially available from MediGuide Ltd., and as generally shown with reference to one or more of U.S. Pat. No. 6,233,476 entitled “Medical Positioning System”; U.S. Pat. No. 7,197,354 entitled “System for Determining the Position and Orientation of a Catheter”; and U.S. Pat. No. 7,386,339 entitled “Medical Imaging and Navigation System,” the disclosures of which are incorporated herein by reference in their entireties.

In a further exemplary embodiment, subsystem 18 may utilize a combination electric field-based and magnetic field-based system as generally shown with reference to U.S. Pat. No. 7,536,218 entitled “Hybrid Magnetic-Based and Impedance Based Position Sensing,” the disclosure of which is incorporated herein by reference in its entirety. In yet still other exemplary embodiments, the subsystem 18 may comprise or be used in conjunction with other commonly available systems, such as, for example and without limitation, fluoroscopic, computed tomography (CT), and magnetic resonance imaging (MRI)-based systems.

In one embodiment wherein subsystem 18 includes an electric field-based system, and as described above, catheter 16 includes a plurality of electrodes 30 configured to both acquire EP data and produce signals indicative of catheter position and/or orientation information (positioning data). Subsystem 18 may use, for example and without limitation, time-division multiplexing or other similar techniques such that positioning data indicative of the position of electrodes 30 is measured intermittently with EP data. Thus, an electric field used to locate electrodes 30 may be activated between measurements of EP data, and electrodes 30 may be configured to measure both EP data and the electric field from subsystem 18, though at different times.

In other embodiments, however, wherein electrodes 30 may not be configured to produce positioning data, catheter 16 may include one or more positioning sensors in addition to electrodes 30. In one such embodiment, catheter 16 may include one or more positioning electrodes configured to generate signals indicative of the 3-D position or location of the positioning electrode(s). Using the position of the positioning electrode(s) along with a known configuration of catheter 16 (e.g., the known spacing between the positioning electrode(s) and electrodes 30) the position or location of each electrode 30 can be determined.

Alternatively, in another embodiment, rather than including an electric-field based system, subsystem 18 includes a magnetic field-based system. In such an embodiment, catheter 16 may include one or more magnetic sensors (e.g., coils) configured to detect one or more characteristics of a low-strength magnetic field. The detected characteristics may be used, for example, to determine a 3-D position or location for the magnetic sensors(s), which may then be used with a known configuration of the catheter 16 to determine a position or location for each electrode 30.

For purposes of clarity and illustration only, subsystem 18 will be described hereafter as comprising an electric field-based system, such as, for example, the ENSITE NAVX™ or VELOCITY™ systems identified above. Further, the description below will be limited to an embodiment of system 10 wherein electrodes 30 are configured to both acquire EP data and produce positioning data. It will be appreciated in view of the above, however, that the present disclosure is not meant to be limited to an embodiment wherein subsystem 18 includes an electric field-based system or electrodes 30 serve a dual purpose or function. Accordingly, embodiments wherein subsystem 18 is other than an electric field-based system, and catheter 16 includes positioning sensors in addition to electrodes 30 remain within the spirit and scope of the present disclosure.

With reference to FIGS. 1 and 6, in this embodiment subsystem 18 may include an electronic control unit (ECU) 1000 and a display device 1002. Alternatively, one or both of ECU 1000 and display device 1002 may be separate and distinct from, but electrically connected to and configured for communication with, subsystem 18. Subsystem 18 may still further include a plurality of patch electrodes 1004, among other components. With the exception of a patch electrode 1004_{B} called a “belly patch,” patch electrodes 1004 are provided to generate electrical signals used, for example, in determining the position and orientation of catheter 16, and in the guidance thereof. Catheter 16 may be coupled to ECU 1000 or subsystem 18 with a wired or wireless connection.

In one embodiment, patch electrodes 1004 are placed orthogonally on the surface of body 14 and are used to create axes-specific electric fields within body 14. For instance, patch electrodes 1004_{X1}, 1004_{X2} may be placed along a first (x) axis. Patch electrodes 1004_{Y1}, 1004_{Y2} may be placed along a second (y) axis, and patch electrodes 1004_{Z1}, 1004_{Z2} may be placed along a third (z) axis. These patches may act as a pair or dipole. In addition or in the alternative, the patches may be paired off an axis or paired in series, e.g., 1004_{X1} is paired with 1004_{Y1}, then 1004_{Y2}, 1004_{Z1}, 1004_{Z2}. In addition, multiple patches may be placed on one axis, e.g., under the patient. Each of the patch electrodes 1004 may be coupled to a multiplex switch 1006. In this embodiment, ECU 1000 is configured, through appropriate software, to provide control signals to switch 10006 to thereby sequentially couple pairs of electrodes 1004 to a signal generator 108. Excitation of each pair of electrodes 1004 generates an electric field within body 14 and within an area of interest such as tissue 12. Voltage levels at the non-excited electrodes 1004, which are referenced to the belly patch 1004_{B}, are filtered and converted and provided to ECU 1000 for use as reference values.

With electrodes 30 electrically coupled to ECU 1000, electrodes 30 are placed within electrical fields that patch electrodes 1004 create in body 14 (e.g., within the heart) when patch electrodes 1004 are excited. Electrodes 30 experience voltages that are dependent on the respective locations between patch electrodes 1004 and the respective positions of electrodes 30 relative to tissue 12. Voltage measurement comparisons made between electrodes 30 and patch electrodes 1004 can be used to determine the position of each electrode 30 relative to tissue 12. Accordingly, ECU 1000 is configured to determine position coordinates (x, y, z) of each electrode 30. Further, movement of electrodes 30 near or against tissue 12 (e.g., within a heart chamber) produces information regarding the geometry of tissue 12.

The information relating to the geometry of the tissue 12 may be used, for example, to generate models and/or maps of anatomical structures that may be displayed on a display device, such as, for example, display device 1002. Information received from electrodes 30 can also be used to display on display device 1002 the location and orientation of the electrodes 30 and/or the tip of catheter 16 relative to tissue 12. Accordingly, among other things, ECU 1000 may provide a means for generating display signals for display device 1002 and for creating a graphical user interface (GUI) on display device 1002. It should be noted that in some instances where the present disclosure refers to objects as being displayed on the GUI or display device 1002, this may actually mean that representations of these objects are being displayed on GUI or the display device 1002.

It should also be noted that while in an exemplary embodiment ECU 1000 is configured to perform some or all of the functionality described above and below, in another exemplary embodiment, ECU 1000 may be separate and distinct from subsystem 18, and subsystem 18 may have another ECU configured to perform some or all of the functionality described herein. In such an embodiment, that ECU could be electrically coupled to, and configured for communication with, ECU 1000. However, for purposes of clarity and illustration only, the description below will be limited to an embodiment wherein ECU 1000 is shared between subsystem 18 and system 10 and is configured to perform the functionality described herein. Still further, despite reference to a “unit,” ECU 1000 may include a number or even a considerable number of components (e.g., multiple units, multiple computers, etc.) for achieving the exemplary functions described herein. In some embodiments, then, the present disclosure contemplates ECU 1000 as encompassing components that are in different locations.

ECU 1000 may include, for example, a programmable microprocessor or microcontroller, or may comprise an application specific integrated circuit (ASIC). ECU 1000 may include a central processing unit (CPU) and an input/output (I/O) interface through which ECU 1000 may receive a plurality of input signals including, for example, signals generated by patch electrodes 1004 and positioning sensors. ECU 1000 may also generate a plurality of output signals including, for example, those used to control display device 1002 and switch 1006. ECU 1000 may be configured to perform various functions, such as those described in greater detail above and below, with appropriate programming instructions or code. Accordingly, in one embodiment, ECU 1000 is programmed with one or more computer programs encoded on a computer-readable storage medium for performing the functionality described herein.

In addition to the above, ECU 1000 may further provide a means for controlling various components of system 10 including, but not limited to, switch 1006. In operation, ECU 1000 generates signals to control switch 1006 to thereby selectively energize patch electrodes 1004. ECU 1000 receives positioning data from catheter 16 reflecting changes in voltage levels and from the non-energized patch electrodes 104. ECU 1000 uses the raw positioning data produced by patch electrodes 104 and electrodes 30, and corrects the data to account for respiration, cardiac activity, and other artifacts using known or hereinafter developed techniques. The corrected data, which comprises position coordinates corresponding to each of electrodes 30 (e.g., (x, y, z)), may then be used by ECU 1000 in a number of ways, such as, for example and without limitation, to create a geometrical anatomical model of an anatomical structure or to create a representation of catheter 16 that may be superimposed on a map, model, or image of tissue 12 generated or acquired by ECU 1000.

ECU 1000 may be configured to construct a geometrical anatomical model of tissue 12 for display on display device 1002. ECU 1000 may also be configured to generate a GUI through which a user may, among other things, view a geometrical anatomical model. ECU 1000 may use positioning data acquired from electrodes 30 or other sensors on distal end 28 or from another catheter to construct the geometrical anatomical model. In one embodiment, positioning data in the form of a collection of data points may be acquired from surfaces of tissue 12 by sweeping distal end 28 of catheter 16 along the surfaces of tissue 12. From this collection of data points, ECU 1000 may construct the geometrical anatomical model. One way of constructing the geometrical anatomical model is described in U.S. patent application Ser. No. 12/347,216 entitled “Multiple Shell Construction to Emulate Chamber Contraction with a Mapping System,” the entire disclosure of which is incorporated herein by reference. Moreover, the anatomical model may comprise a 3-D model or a two-dimensional (2-D) model. As will be described in greater detail below, a variety of information may be displayed on the display device 1002, and in the GUI displayed thereon, in particular, in conjunction with the geometrical anatomical model, such as, for example, EP data, images of catheter 16 and/or electrodes 30, metric values based on EP data, HD surface maps, and HD composite surface maps.

To display the data and images that are produced by ECU 1000, display device 1002 may include one or more conventional computer monitors other display devices well known in the art. It is desirable for display device 1002 to use hardware that avoids aliasing. To avoid aliasing, the rate at which display device 1002 is refreshed should be at least as fast as the frequency with which ECU 1000 is able to continuously compute various visual aids, such as, for example, HD surface maps.

As described above, the plurality of electrodes 30 disposed at distal end 28 of catheter 16 are configured to acquire EP data. The data collected by the respective electrodes 30 may be collected simultaneously. In one embodiment, EP data may include at least one electrogram. An electrogram indicates the voltage measured at a location (e.g., a point along tissue 12) over a period of time. By placing a high density of electrodes 30 on distal end 28, ECU 1000 may acquire a set of electrograms measured from adjacent locations in tissue 12 during the same time period. The adjacent electrode 30 locations on distal end 28 may collectively be referred to as a “region.”

ECU 1000 may also acquire times at which electrograms are measured, the positions from which electrograms are measured, and the distances between electrodes 30. As for timing data, ECU 1000 may track, maintain, or associate timing data with the voltages of each electrode 30 as measured. In addition, the 3-D position coordinates of each electrode 30 as it measures voltages may be determined, for example, as described above by visualization, navigation, and mapping subsystem 18. ECU 1000 may be configured to continuously acquire position coordinates of electrodes 30, especially when electrodes 30 are measuring EP data. Because ECU 1000 may know the spatial distribution of electrodes 30 of each distal end 28 configuration (e.g., matrix-like, spiral, basket, etc.), ECU 1000 may recognize from the position coordinates of electrodes 30 which configuration of distal end 28 is deployed within a patient. Furthermore, the distances between electrodes 30 may be known by ECU 1000 because electrodes 30 may be precisely and strategically arranged in a known spatial configuration. Thus, if distal end 28 is not deformed, a variety of analyses may use the known distances between electrodes 30 without having to obtain the coordinate positions from the subsystem 18 to solve for the distances between electrodes 30.

With ECU 1000 having voltage, timing, and position data corresponding to respective electrodes 30 in addition to the known electrode 30 spatial configuration, many comparative temporal and spatial analyses may be performed, as described below. Some of these analyses lead to creation of HD surface maps representing activation patterns from tissue 12, which are possible in part because of the high density of electrodes 30 at distal end 28 of shaft 24. By providing a high density of electrodes at distal end 28, the accuracy and resolution of HD surface maps produced by system 10 are enhanced.

With respect to capturing or collecting EP data measured by the high density of electrodes 30, in one embodiment, ECU 1000 may be programmed to continuously record and analyze data in real-time or near real-time. In another embodiment, a user may specify through a user input device a time window (e.g., 200 ms, 30 seconds, 10 minutes etc.) during which ECU 1000 may capture data measured from electrodes 30. The user input device may include, for example and without limitation, a mouse, a keyboard, a touch screen, and/or the like. It should be noted that in one embodiment, electrodes 30 may continuously measure voltages along tissue 12, and ECU 1000 may selectively capture or record such voltages from electrodes 30. In still another embodiment, electrodes 30 measure voltages in accordance with a sampling rate or command from ECU 1000. Once distal end 28 of shaft 24 is positioned near or along tissue 12 as desired, the user could prompt a trigger for the time window. The user may configure the trigger for the time window to correspond, for example, to a particular cardiac signal or the expiration of a timer. To illustrate, trigger could be set so ECU 1000 records data from electrodes 30 before, during, and after an arrhythmia breakout or disappearance. One possible way to capture the data occurring just prior to the particular cardiac signal would be to use a data buffer that stores data (which may later be obtained) for an amount of time.

ECU 1000 may be configured to recognize particular cardiac signals to trigger the time window. To that end, electrodes 30 may constantly measure EP data when positioned near tissue 12. This may be the case even if the user has not prompted the trigger for the time window. For example, ECU 1000 may recognize that distal end 28 is near tissue 12 inside body 14 based on the continuous measurements in the range of voltages that are expected near tissue 12. Or ECU 1000 may, for example, be configured to constantly monitor voltages from electrodes 30 when ECU 1000 is powered “on.” In any event, ECU 1000 may continuously acquire EP data and continuously assess patterns and characteristics in the EP data. For example, metrics based on EP data include, for example, local activation time (LAT), depolarization amplitude voltage (e.g., peak-to-peak amplitude (PP)), complex fractionated electrogram (CFE) activity, dominant frequency (DF), Fast Fourier Transform (FFT) ratio, activation potential, diastolic potential, and late potential. U.S. Pat. No. 9,186,081 entitled “System and Method for Diagnosing Arrhythmias and Directing Catheter Therapies”, the disclosure of which is incorporated herein by reference in its entirety, discloses multiple examples of metrics based on EP data.

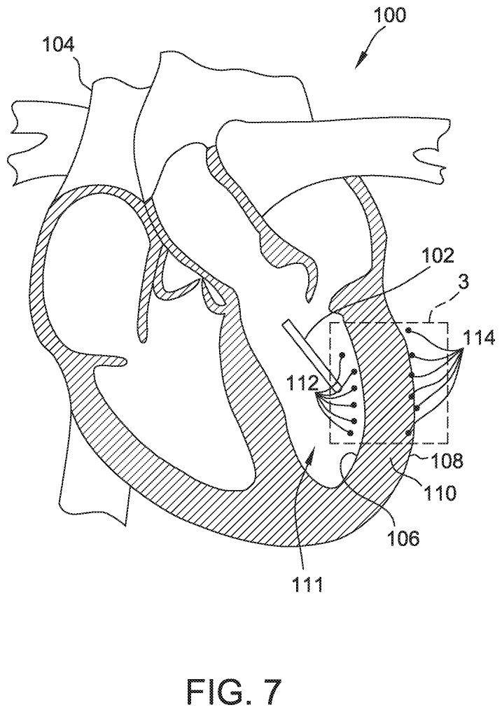

FIG. 7 illustrates one exemplary embodiment of a system 100 for measuring transmural activation using a point-to-point correlation technique. As described herein, the transmural activation is determined using a computing device, such as ECU 1000 (shown and described in connection with FIG. 6). In system 100, a catheter 102 (e.g., catheter 16) is positioned at different points inside and outside a subject's heart 104, as described in detail herein. In the embodiment shown in FIG. 7, catheter 102 is used to measure transmural activation between an endocardial surface 106 and an epicardial surface 108 of a cardiac wall 110 defining the left ventricle 111 of heart 104. Alternatively, catheter 102 may be used to measure transmural activation across other anatomical structures.

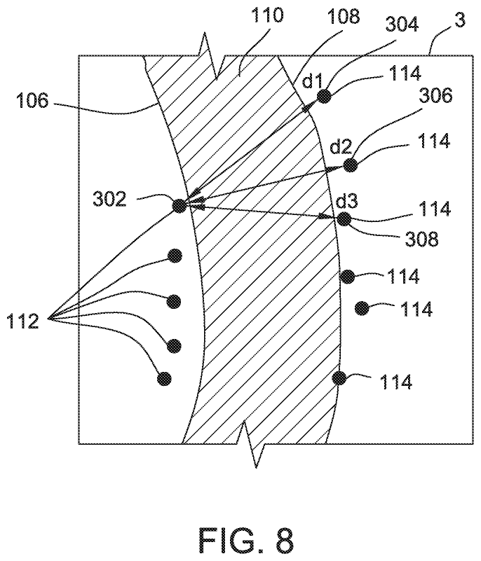

FIG. 8 is an enlarged view of a portion 3 of cardiac wall 110. As shown in FIGS. 7 and 8, to measure transmural activation across cardiac wall 110, catheter 102 is positioned at and acquires electrogram data and positioning data (i.e., indicating the location of catheter 102) at a plurality of endocardial points 112 and epicardial points 114. Endocardial points 112 are points on or proximate endocardial surface 106, and epicardial points 114 are points on or proximate epicardial surface 108. In this embodiment, catheter 102 acquires electrogram data and positioning data at six endocardial points 112 and six epicardial points 114. Alternatively, electrogram data and positioning data may be acquired at any suitable number of endocardial and epicardial points 112 and 114. In one embodiment, the positioning data (and thus the positions) of the endocardial and epicaridal points 112 and 114 is acquired using the techniques described above in connection with FIGS. 1-6. Alternatively, the positioning data may be obtained using any suitable methods.

Based on the electrogram data and positioning data acquired at endocardial points 112 and epicardial points 114, one or more transmural activation times can be calculated for electrical activations across cardiac wall 110, as described herein. The transmural activation times may be calculated by a computing device, such as ECU 1000 (shown in FIG. 6 and described above in detail). Transmural activation may be hampered, for example, by intramural scarring. Accordingly, longer transmural activation times may facilitate locating intramural scars.

Specifically, in one embodiment, each endocardial point 112 is paired with an epicardial point 114 that is located the shortest distance from that endocardial point 112. The ‘distance’ may be an actual physical distance, or some other distance metric. For example, as shown in FIG. 8, a first endocardial point 302 is located a first physical distance d1 from a first epicardial point 304, a second physical distance d2 from a second epicardial point 306, and a third physical distance d3 from a third epicardial point 308. As the third physical distance d3 is the shortest distance, first endocardial point 302 is paired with third epicardial point 308. In other embodiments, the ‘distance’ between endocardial points 112 and epicardial points 114 may be at least partially defined by a conduction time (i.e., the electrical time delay) between two considered points. Fiber orientations between the two points may impact the conduction time, such that two points out may have the shortest conduction time between one another without being the physically closest points. Accordingly, the ‘distance’ between endocardial points 112 and epicardial points 114, as used herein, may be defined based on a physical distance, a conduction time, or some combination of physical distance and conduction time.

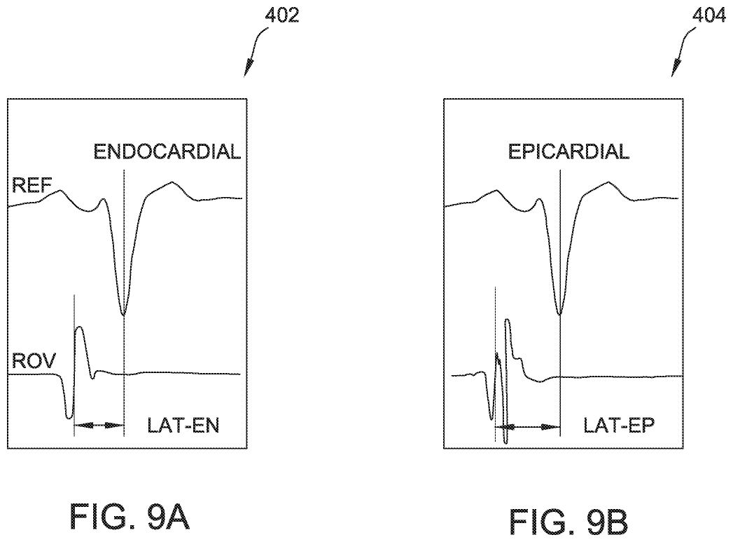

For a pair including one endocardial point 112 and one epicardial point 114, an activation time can then be calculated based on the electrogram data acquired for those two points. For example, FIG. 9A shows a first electrogram 402 measured at an endocardial point 112, and FIG. 9B shows a second electrogram 404 measured at an epicardial point 114. As shown in FIGS. 9A and 9B, an endocardial activation time (LAT-EN) can be determined from first electrogram 402, and an epicardial activation time (LAT-EP) can be determined from second electrogram 404.

For example, the REF signal represents a surface ECG channel recording that is acquired simultaneously with but independently from the electrograms acquired by catheter 102 at endocardial and epicardial points 112 and 114. Therefore, the REF signal can be used as a reference for electrograms acquired at endocardial and epicardial points 112 and 114. Different features (e.g., a maximum peak, a minimum peak, a maximum slope, etc.) of the REF signal and electrograms may be used as references to measure LAT-EN and LAT-EP. In one embodiment, for example, LAT-EN and LAT-EP are the time intervals between the minimum peak on the REF signal and the maximum slope on the respective electrograms. Alternatively, any suitable features may be used to determine LAT-EN and LAT-EP. The transmural activation time is then given by the following equation:

$$Transmural Activation Time = \frac{EN Activation Time - EP Activation Time}{Tissue Thickness}$$

where EN Activation Time is LAT-EN, EP Activation Time is LAT-EP, and Tissue Thickness is the distance between the endocardial point 112 and the epicardial point 114 in the pair.

To ensure proper pairing of endocardial and epicardial points 112 and 114, point densities of endocardial and epicardial points 112 and 114 should be sufficiently dense. Accordingly, in some embodiments, density parameters representing endocardial point density and epicardial point density are calculated and compared to a threshold (e.g., by a computing device) to determine whether the point density is sufficient.

For example, in one embodiment, the density parameters are average point-to-point distances for the endocardial and epicardial points 112 and 114, respectively. That is, the endocardial density parameter would be the average point-to-point distance between endocardial points 112, and the epicardial density parameter would be the average point-to-point distance between epicardial points 114. In another embodiment the density parameters are maximum point-to-point distances for the endocardial and epicardial points 112 and 114, respectively. That is, the endocardial density parameter would be the largest point-to-point distance between any two endocardial points 112, and the epicardial density parameter would be the largest point-to-point distance between any two epicardial points 114.

The density parameters can then be compared to a threshold value. In one embodiment, the threshold value is 50% of a discrete or average measured tissue thickness of cardiac wall 110. If the density parameters are each less than the threshold value, the point densities of endocardial and epicardial points 112 and 114 are considered to be sufficient. Further, in some embodiments, normalization with respect to the measured tissue thickness may be performed to account for varying endocardial to epicardial point distances and the influence of those distances on calculated activation times. Alternatively, the point densities of endocardial and epicardial points 112 and 114 may be assessed using any suitable techniques. If the point densities of endocardial or epicardial points 112 and 114 are determined to be insufficient, an appropriate alert may be automatically generated (e.g., by subsystem 18).

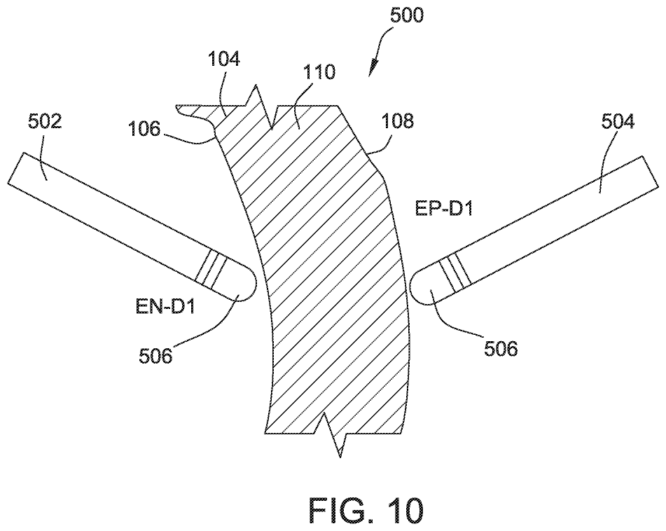

FIG. 10 illustrates one exemplary embodiment of a system 500 for measuring transmural activation using catheter-to-catheter techniques. In this embodiment, the transmural activation is determined using a computing device, such as ECU 1000 (shown in and described in connection with FIG. 6). In system 500, a first catheter 502 is positioned inside heart 104 adjacent endocardial surface 106, and a second catheter 504 is positioned outside heart 104 adjacent epicardial surface 108. First and second catheters 502 and 504, may be, for example, catheter 16 (shown in FIG. 1). In this embodiment, first and second catheters 502 and 504 are used to measure transmural activation between endocardial surface 106 and epicardial surface 108 of cardiac wall 110. Alternatively, first and second catheters 502 and 504 may be used to measure transmural activation across other anatomical structures.

First and second catheters 502 and 504 may be used to measure transmural activation using activation mapping or pace mapping, both of which are described herein. Further, first and second catheters 502 and 504 may be single-electrode catheters or multi-electrode catheters (e.g., high density mapping catheters).

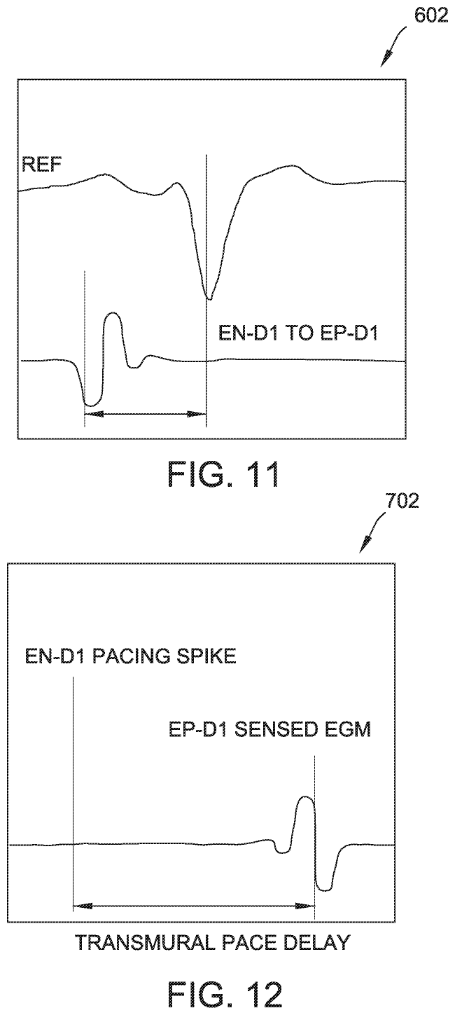

For catheter-to-catheter activation mapping, in one embodiment, bipolar intracardiac electrograms (IEGMs) are collected in real-time (i.e., substantially simultaneously) using at least one electrode 506 on each of first and second catheters 502 and 504. For example, FIG. 11 shows an electrogram 602 measured by first catheter 502 or second catheter 504.

Based on the collected IEGMs, the transmural activation time is calculated (e.g., by a computing device, such as ECU 1000 (shown and described above in connection with FIG. 6) as the time difference between when a particular electrogram feature (e.g., the Q wave, or any other suitable electrogram feature) is detected at first catheter 502 and second catheter 504.

In some embodiments, the transmural activation time is only calculated or recorded when a distance between the at least one recording electrode 506 on first catheter 502 (EN-D1) and the at least one recording electrode 506 on second catheter 504 (EP-D1) is less than a predetermined threshold distance. The predetermined threshold distance may be user defined, or may be calculated based on a tissue thickness (e.g., measured using CT data). For example, in one embodiment, the predetermined threshold distance is approximately 10 millimeters (mm).

Because the IEGMs are collected in real-time by first and second catheters 502 and 504, parameters other than transmural activation times may also be calculated. For example, in some embodiments, EGM fragmentation and/or voltages may be calculated. Further, in some embodiments a two-dimensional transmural activation vector can be calculated, where one component of the vector is the transmural activation time, and the other component of the vector is the distance between the electrodes 506 on first and second catheters 502 and 504. The two-dimensional transmural activation vector may also be similarly calculated for the other embodiments described herein.

As another example, in some embodiments, endocardial-epicardial bipolar impedance measurements may be acquired to provide further information on the transmural tissue properties. As yet another example, properties of a bipolar transmural electrogram (e.g., a bipolar signal obtained by subtracting the endocardial unipolar signal from the epicardial unipolar signal), such as electrogram duration, number of peaks, and local activation may be acquired. Relatively long electrogram duration and/or relatively large numbers of peaks generally indicated slow conduction in the transmural direction. Transmural recordings may also be used to facilitate eliminating far-field signal components and identifying local activation signals. For example, during ventricular tachycardia, a far-field signal at a critical isthmus zone may be much larger than a local activation signal, even in bipolar electrograms. However, bipolar EGMs collected from endocardial surface 106 and epicardial surface 108 at the same approximate location may be used to reduce the far-field component.

For catheter-to-catheter pace mapping, in one embodiment, a pacing pulse is delivered using one of first and second catheters 502 and 504, and the pacing pulse is sensed using the other of first and second catheters 502 and 504. For example, the electrode 506 on first catheter 502 (EN-D1) may generate a pacing pulse to be detected by the electrode 506 on second catheter 504 (EP-D1). Alternatively, the electrode 506 on second catheter 504 (EP-D1) may generate a pacing pulse to be detected by the electrode 506 on first catheter 502 (EN-D1). In some embodiments, both first catheter 502 and second catheter 504 may deliver a pacing pulse to be sensed by the other catheter.

FIG. 12 shows an electrogram 702 measured by second catheter 504 when a pacing pulse is delivered by first catheter 502. The delay time (also referred to as a transmural pace delay) between the delivery of the pacing pulse by one catheter and the sensing of the pacing pulse by the other catheter corresponds to the transmural activation time. The delay time (and thus, the transmural activation time) may be calculated by a computing device, such as subsystem 18 (shown in FIG. 1).

Similar to the activation mapping described above, in some embodiments, the transmural activation time is only calculated or recorded when a distance between the electrode 506 on first catheter 502 (EN-D1) and the electrode 506 on second catheter 504 (EP-D1) is less than a predetermined threshold distance. The predetermined threshold distance may be user defined, or may be calculated based on a tissue thickness (e.g., measured using CT data). For example, in one embodiment, the predetermined threshold distance is approximately 10 millimeters (mm).

Transmural activation times determined using systems 100 and 500 may be displayed, for example, on a display device coupled to or integrated within subsystem 18, such as display device 1002 (shown and described in connection with FIG. 6). Those of skill in the art will appreciate that the methods described in association with FIGS. 7-12 could implemented using image integration and/or magnetic navigation systems (e.g., the EnSite™ Precision™ system commercially available from Abbott Laboratories, and generally shown with reference to U.S. Pat. No. 7,263,397 entitled “Method and Apparatus for Catheter Navigation and Location and Mapping in the Heart”, the entire disclosure of which is incorporated herein by reference), such as those described above in connection with FIGS. 2-6 to facilitate more accurate determinations of the endocardial/epicardial surface representations and tissue thickness measurements. Further, catheters used in systems 100 and 500 may be single or multi-electrode mapping catheters.

Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

What is claimed is:

1. A system for measuring transmural activation times between an endocardial surface and an epicardial surface, the system comprising:
at least one catheter comprising at least one electrode, the at least one catheter configured to acquire electrogram data and positioning data proximate at least one of the endocardial surface and the epicardial surface; and
a computing device communicatively coupled to the at least one catheter, the computing device configured to determine transmural activation times based on the acquired electrogram data and positioning data.

2. The system of claim 1, wherein the at least one catheter is configured to acquire electrogram data and positioning data for a plurality of endocardial points and a plurality of epicardial points.

3. The system of claim 2, wherein the computing device is configured to:
pair each of the plurality of endocardial points with a corresponding epicardial point of the plurality of epicardial points; and
for at least one pair, calculate a transmural activation time based on the electrogram data for the endocardial point and epicardial point in the pair.

4. The system of claim 3, wherein to pair each of the plurality of endocardial points with a corresponding epicardial point, the computing device is configured to pair each endocardial point with the epicardial point that is located the shortest distance from that endocardial point.

5. The system of claim 2, wherein the computing device is further configured to:
calculate a first density parameter for the plurality of endocardial points;
calculate a second density parameter for the plurality of epicardial points;
compare the first and second density parameters to a threshold value; and
generate an alert when at least one of the first and second density parameters exceeds than the threshold value.

6. The system of claim 1, wherein the at least one catheter comprises:
a first catheter positioned proximate the endocardial surface; and
a second catheter positioned proximate the epicardial surface.

7. A method for measuring transmural activation times between an endocardial surface and an epicardial surface, the system comprising:
acquiring electrogram data and positioning data using at least one catheter, the at least one catheter including at least one electrode proximate at least one of the endocardial surface and the epicardial surface; and
determining, using a computing device communicatively coupled to the at least one catheter, at least one transmural activation time based on the acquired electrogram data and positioning data.

8. The method of claim 7, wherein acquiring electrogram data and positioning data comprises acquiring electrogram data and positioning data for a plurality of endocardial points and a plurality of epicardial points.

9. The method of claim 8, wherein determining at least one transmural activation time comprises:
pairing each of the plurality of endocardial points with a corresponding epicardial point of the plurality of epicardial points; and
for at least one pair, calculating a transmural activation time, using the computing device, based on the electrogram data for the endocardial point and epicardial point in the pair.

10. The method of claim 9, wherein pairing each of the plurality of endocardial points with a corresponding epicardial point comprises pairing each endocardial point with the epicardial point that is located the shortest distance from that endocardial point.

11. The method of claim 8, further comprising:
calculating, using the computing device, a first density parameter for the plurality of endocardial points;
calculating, using the computing device, a second density parameter for the plurality of epicardial points;
comparing, using the computing device, the first and second density parameters to a threshold value; and
generating, using the computing device, an alert when at least one of the first and second density parameters exceeds the threshold value.

12. The method of claim 7, wherein the at least one catheter includes a first catheter proximate the endocardial surface and a second catheter proximate the epicardial surface.

13. The method of claim 12, wherein determining at least one transmural activation time comprises determining a transmural activation time as the time difference between when a particular electrogram feature is detected at the first catheter and when the particular electrogram feature is detected at the second catheter.

14. The method of claim 12, further comprising:
determining, using the computing device, a distance between a first electrode on the first catheter and a second electrode on the second catheter;
comparing, using the computing device, the determined distance to a predetermined threshold distance; and
determining, using the computing device, at least one transmural activation time only when the determined distance is less than the predetermined threshold distance.

15. The method of claim 12, further comprising:
delivering a pacing pulse using one of the first catheter and the second catheter; and
detecting the delivered pacing pulse using the other of the first catheter and the second catheter, wherein determining at least one transmural activation time comprises determining a transmural activation time as the time delay between the delivery of the pacing pulse by the one of the first catheter and the second catheter and the detection by the other of the first catheter and the second catheter.

16. The method of claim 12, further comprising:
calculating, using the computing device, at least one additional parameter other than transmural activation times based on the acquired electrogram data and positioning data.

17. A computing device for measuring transmural activation times between an endocardial surface and an epicardial surface, the computing device configured to:
receive electrogram data and positioning data from at least one catheter communicatively coupled to the computing device, the at least one catheter including at least one electrode proximate at least one of the endocardial surface and the epicardial surface; and
determine at least one transmural activation time based on the acquired electrogram data and positioning data.

18. The computing device of claim 17, wherein to receive electrogram and positioning data, the computing device is configured to receive electrogram data and positioning data for a plurality of endocardial points and a plurality of epicardial points.

19. The computing device of claim 18, wherein the computing device is further configured to:
calculate a first density parameter for the plurality of endocardial points;
calculate a second density parameter for the plurality of epicardial points;
compare the first and second density parameters to a threshold value; and
generate an alert when at least one of the first and second density parameters exceeds the threshold value.

20. The computing device of claim 17, wherein to receive electrogram and positioning data, the computing device is configured to receive electrogram data and positioning data from a first catheter proximate the endocardial surface and a second catheter proximate the epicardial surface.
